(19) United States
(12) Patent Application Publication
(10) Pub. No.: US 2004/0010164 A1
(43) Pub. Date: Jan. 15, 2004

(54) Process for the production of canthaxanthin

(75) Inventors: Frank Dubner, Friedberg (DE); Jurgen Bilz, Freigericht (DE); Christoph Weckbecker, Grundau-Lieblos (DE); Klaus Huthmacher, Gelnhausen (DE)

Correspondence Address: FITCH, EVEN, TABIN & FLANNERY, SUITE 401L, 1801 K STREET, NW, WASHINGTON, DC, 20006-1201, US

(73) Assignee: Degussa AG

(21) Appl. No.: 10/455,806

(22) Filed: Jun. 6, 2003

(30) Foreign Application Priority Data
Jun. 11, 2002 (DE) 102 25 856.2

(51) Int. Cl. C07C 45/28

(52) U.S. Cl. 568/357

(57) ABSTRACT

The present invention relates to a process for the production of canthaxanthin by oxidation of beta-carotene or carotinoids derived therefrom which have conjugated double bonds.

### CROSS REFERENCE TO RELATED APPLICATIONS

The present application claims priority to German Application No. 102 25 856.2, filed Jun. 11, 2002, which is incorporated in its entirety herein by reference.

### FIELD OF THE INVENTION

The present invention is directed to a process for the production of canthaxanthin by the oxidation of beta-carotene or carotinoids derived therefrom having conjugated double bonds.

### BACKGROUND OF THE INVENTION

Canthaxanthin is one of many natural pigments and is found in a large number of animals and plants. It has been widely used as a red dye both in aquaculture and poultry rearing.

Known processes for the formation of canthaxanthin from beta-carotene use either salts of chloric or bromic acid (DE 2534805), salts of periodic acid (U.S. Pat. No. 3,646,149) or manganese oxides (Lu, et al., Fine Chemicals, China, 17:74-76 (2000)) as the oxidizing agent. However, long reaction times or a large excess of oxidizing agent are needed for complete conversion. These processes are therefore uneconomical and raise concerns regarding safety.

In example 1 of U.S. Pat. No. 3,646,149, beta-carotene is reacted with 4 equivalents of sodium periodate and 2 mol percent iodine in a two-phase mixture of chloroform and water. After a reaction time of two hours, canthaxanthin is obtained by precipitation at a yield of 33.7%. No details are given of the desired all-trans content.

Example 1 of DE 2534805 discloses the oxidation of beta-carotene with ten equivalents of sodium chlorate and the addition of catalytic quantities of iodine. The reaction is complete only after 36 hours at room temperature and, after precipitation, yields 69.5% canthaxanthin with an all-trans content of 82%. Example 2 suggests that the yield can be increased to 78.5% if no less than 30 equivalents of sodium chlorate are used. The use of catalysts other than iodine, such as e.g. potassium iodide or oxides of elements from Group Va, VIa or VIIa produced poorer results.

In EP-A-1059290 the reaction time is successfully reduced to 2 hours with comparable yields by using the catalysts iodine and potassium iodide at the same time. However, here too, 15 mol-equivalents of sodium chlorate (which is fire-promoting and tends towards explosiveness at high temperatures) are needed. At the end of the reaction a mixture is obtained that contains 76% canthaxanthin with an all-trans content of 52%.

### OBJECT OF THE INVENTION

The object of the present invention is to provide a process that requires no fire-promoting chemicals and can nevertheless be carried out in short reaction times.

### SUMMARY OF THE INVENTION

The invention is directed to a process for the production of canthaxanthin in which the direct oxidation of beta-carotene, or one of the precursors of canthaxanthin described above, can be carried out with commercial salts of hypohalogen acids or released hypohalogen acids, such as for example HOCl, HOI or HOBr, and their decomposition products, with complete conversion and good selectivity.

In particular, the invention is directed to a process for the production of canthaxanthin by the oxidation of beta-carotene or other oxidation intermediates, in the presence of an oxidizing agent, a catalyst and an inert, organic solvent. The oxidizing agent used may be a salt of a hypohalogen acid, a hypohalogen acid released “in situ” by the acidification of an aqueous solution of such a salt, or a decomposition product thereof.

Compounds that may be oxidized include: retro-dehydrocarotene; echinenone; isozeaxanthin; isocryptoxanthin; and 4′-hydroxyechinenone. In preferred embodiments, the oxidizing agent is selected from the group consisting of: a) an earth alkali-, alkali- or ammonium salt of hypochlorous acid, hypobromous acid or hypoiodous acid; b) a free acid produced “in situ” by the acidification of an aqueous solution of an earth alkali-, alkali- or ammonium salt of hypochlorous acid, hypobromous acid or hypoiodous acid; and c) a decomposition product derived from the salt of a) or the acid of b). One particularly preferred oxidizing agent is produced “in situ” from sodium hypochlorite.

The process described above should be carried out at a temperature of between −10° C. and 70° C., and preferably at between 0° C. and 10° C. Inert, organic solvents that may be used include a chlorinated aliphatic hydrocarbon, an aromatic hydrocarbon, a dialkyl ether or a carbon disulfide. Examples of preferred solvents are chloroform, dichloro-methane; 1,1-dichloroethane, 1,2-dichloroethane, 1,1,2-trichloroethane, 1,1,2,2-tetrachloro-ethane, 1,2-dichloroethylene, 1,1,2-trichloro-ethylene, benzene, toluene nitrobenzene, chlorobenzene, diethylether, and di-n-propylether.

Preferred catalysts include: a) a halogen, an oxide or an oxo acid of an element of Group Va, VIa, or VIIa of the periodic table, or a salt thereof; or b) an oxide of an element of Group VIII of the periodic table, or a salt thereof. Particularly preferred catalysts include: a) elemental chlorine, bromine or iodine; b) an alkali, earth alkali or ammonium salt of the corresponding hydrohalic acid of elemental chlorine, bromine or iodine; c) a mixture of any of the elements of a) and/or the salts of b); or d) osmium tetroxide.

The molar ratio of oxidizing agent to beta-carotene or a carotinoid in reactions should be between 1:1 and 10:1 mol., and preferably between 1:1 and 5:1 mol. Oxidation should be performed at a pH of 4 to 8. Reaction time should be 0.1 to 10 hours and preferably about 1 hour.

### DETAILED DESCRIPTION OF THE INVENTION

The present invention is concerned with a process for the production of canthaxanthin by the oxidation of compounds such as beta-carotene, retro-dehydrocarotene, echinenone, isozeaxanthin, isocryptoxanthin, 4′-hydroxyechinenone or other oxidation intermediates. Oxidizing agents include salts of hypohalogen acids or released hypohalogen acids, such as, for example, HOCl or HOBr, and their decomposition products. Reactions should be performed at a pH of 4-8, in the presence of halogens, such as chlorine, bromine or iodine or of oxides or oxo acids of the elements of Groups Va, VIa, or VIIa, of the periodic system or of oxides of Group VIII of the periodic system, or the salts thereof. Reactions should also include an organic solvent that is inert towards the reaction components at low temperatures.

The present process has several advantages over those that have been used in the past. These include the ability to use chlorine bleach solution (NaOCl solution in water), which is inexpensive and preferable from the point of view of safety, the use of only 4 equivalents of oxidizing agent and very short reaction times. The process is thus very economical.

The starting materials are oxidized in the form of solutions. Solutions of 1 to 40 g of the carotinoid to be oxidized in 1 liter of an organic solvent inert towards the reaction components and not miscible with water, are preferred. Suitable inert, organic solvents include: chlorinated aliphatic hydrocarbons (for example chloroform, dichloromethane, 1,1-dichloroethane, 1,2-dichloroethane, 1,1,2-trichloroethane, 1,1,2,2-tetrachloroethane, 1,2-dichloroethylene, and 1,1,2-trichloroethylene), aromatic hydrocarbons (for example benzene, toluene, nitrobenzene or chlorobenzene), dialkylethers (for example diethylether, di-n-propylether or carbon disulfide). Chloroform and dichloromethane are particularly suitable solvents.

Suitable oxidizing agents include salts of hypochlorous acid or hypobromous acid (in particular the earth alkali-, alkali- or ammonium salts) or the free acids produced “in situ” and their decomposition products, which form when acidifying the salt solutions. Examples of oxidizing agents are sodium hypochlorite, potassium hypochlorite, sodium hypobromite, potassium hypobromite, sodium hypoiodite, and potassium hypoiodite. Preferred oxidizing agents are sodium hypobromite and most preferably, sodium hypochlorite. Decomposition products are, for example, free halogen acids, such as chloric, bromic or iodic acids at a pH below 7, or their salts, such as chlorates, bromates or iodates at a pH above 7.

The oxidizing agents may be added to the reaction mixture in the form of aqueous solutions at a concentration of 1 to 15 wt. %. The molar ratio of oxidizing agent to starting substance should be in the range of 1:1 to 10:1, and preferably in the range of 1:1 to 5:1 mol. A more than 10-fold molar excess of oxidizing agent leads to losses in yield.

As indicated above, oxidation may be catalyzed by: the halogens chlorine, bromine or iodine; by oxides or oxo acids of selenium; by the oxides or oxo acids of elements of groups Va, VIa, or VIIa of the periodic system; oxides of group VIII of the periodic system; or by their salts. Suitable catalysts are, for example: selenium dioxide; selenious acid or its salts; selenic acid or its salts; vanadium pentoxide; vanadates; polyvanadic acids or their salts; heteropolyacids of vanadium, in particular with the elements tungsten, molybdenum and phosphorus, or their salts; molybdenum trioxide; molybdates, in particular ammonium molybdate; polymolybdates; heteropolyacids of molybdenum, in particular with the elements vanadium or phosphorus; tungsten trioxide; tungstates; polytungstic acids or their salts; heteropolyacids of tungsten, in particular with the elements vanadium and phosphorus or their salts; manganese dioxide; nickel oxide; osmium tetroxide; or mixtures thereof. Preferred catalysts are the free halogens chlorine, bromine, iodine and the alkali, earth alkali or ammonium salts of the corresponding hydrohalic acids, such as, for example, sodium chloride, -bromide or -iodide, potassium chloride, -bromide or -iodide, or ammonium chloride, -bromide or -iodide, and osmium tetroxide. Elemental iodine and potassium iodide or a mixture of both are particularly suitable as a catalyst.

The catalyst can be added in pure form or in solution, e.g., in the solvent that was used to dissolve the carotinoid to be oxidized, or in water. Alternatively, the catalyst can be formed “in situ.” The quantity of catalyst is usefully 0.1 to 10 wt. %, and preferably 1 to 5 wt. %, in relation to the carotinoid to be oxidized.

Due to the thermal sensitivity of the substances, oxidation is carried out at low temperatures, such as, for example, −10° C. to 70° C., and preferably 0° C. to 10° C. To set the desired pH value, an acid, for example sulfuric acid, hydrochloric acid, acetic acid, or buffer mixtures, may be used. Depending on the conditions selected, reactions may last for 0.1 to 10 hours, and preferably 0.1 to 5 hours. In a particularly advantageous embodiment, 1 hour is needed to obtain the optimum yield of canthaxanthin.

To prevent canthaxanthin from being oxidized by the effects of atmospheric oxygen, reactions should be carried out in an inert gas atmosphere. Inert gases that are suitable for use in the reactions are argon, neon, helium, carbon dioxide, and, in particular, nitrogen.

### EXAMPLES

### Example 1

A solution of 55 mg iodine in 30 ml dichloromethane is added to 4 g beta-carotene in 270 ml dichloromethane in a nitrogen atmosphere at 0-5° C. 20 ml chlorine bleach solution (NaOCl, ca 12% active chlorine content) is then added drop by drop at this temperature within 30 minutes. Prior to addition, the chlorine bleach solution is slightly acidified (pH 6) with 1 N H₂SO₄ and is used within 10 minutes. After addition, the mixture is stirred for a further 30 minutes at 0° C. During this time, the organic phase darkens increasingly. The mixture is diluted with water and the phases are separated. The organic phase is washed with water, dried over sodium sulfate and solvent is removed in a vacuum. 4.8 g of the raw product with a canthaxanthin content of 80 surface percent is obtained. The all-trans content of the canthaxanthin is 71% as measured by HPLC. For isomerization, 25 ml acetone and 40 mg iodine are added to the residue, which is refluxed until a z-isomer signal no longer occurs in HPLC.

The canthaxanthin can be separated out of the reaction mixture by the usual methods for the separation of constituents of a mixture. For example, the solvent can be removed by distillation at reduced pressure and the canthaxanthin can be isolated by recrystallization.

### Example 2

A solution of 40 mg sodium iodide in 10 ml water is added to 4 g beta-carotene in 270 ml dichloromethane in a nitrogen atmosphere at 0-5° C. 20 ml chlorine bleach solution (NaOCl, ca 12% active chlorine content) is then added drop by drop at this temperature within 30 minutes. Before addition, the chlorine bleach solution is slightly acidified (pH 6) with 1 N H₂SO₄ and used within 10 minutes. After addition, the mixture is stirred for a further 30 minutes at 0° C. During this time, the color of the organic phase darkens increasingly. The mixture is diluted with water and the phases are separated. The organic phase is washed with water, dried over sodium sulfate and the solvent is removed in a vacuum. 4.9 g of the raw product with a canthaxanthin content of 72 surface percent is obtained. The all-trans content of the canthaxanthin is 65% as measured by HPLC. For isomerization, 25 ml acetone and 40 mg iodine are added to the residue, which is refluxed until a z-isomer signal no longer occurs in HPLC.

HPLC Analysis conditions:

| | |
|---|---|
| Column: | Inertsil C-8 5μ 250 × 4.6, 40° C., 1.0 ml/min |
| Solvent: | Water/Methanol/THF from 10/1/9 to 9/2/29 |
| Detector: | UV/VIS 450 nm |

All references cited herein are fully incorporated by reference. Having now fully described the invention, it will be understood by those of skill in the art that the invention may be performed within a wide and equivalent range of conditions, parameters and the like, without effecting the spirit or scope of the invention or any embodiment thereof.

What is claimed is:

1. A process for the production of canthaxanthin, comprising oxidizing beta-carotene or a carotinoid derived therefrom and having conjugated double bonds, wherein oxidation is carried out using a reaction mix comprising:
a) an oxidizing agent, wherein said oxidizing agent is a hypohalogen acid, a salt of a hypohalogen acid or a decomposition product of said hypohalogen acid or salt of a hypohalogen acid;
b) a catalyst; and
c) an inert, organic solvent.

2. The process of claim 1, wherein said oxidizing agent is selected from the group consisting of:
a) an earth alkali-, alkali- or ammonium salt of hypochlorous acid, hypobromous acid or hypoiodous acid;
b) a free acid produced “in situ” by the acidification of an aqueous solution of an earth alkali-, alkali- or ammonium salt of hypochlorous acid, hypobromous acid or hypoiodous acid;
c) a decomposition product derived from the salt of paragraph a) or the acid of paragraph b).

3. The process of either claim 1 or 2, wherein said oxidizing agent is produced “in situ” from sodium hypochlorite.

4. The process of claim 1, wherein the compound oxidized is selected from the group consisting of: beta-carotene; retro-dehydrocarotene; echinenone; isozeaxanthin; isocryptoxanthin; and 4′-hydroxyechinenone.

5. The process of claim 1, wherein said process is carried out at a temperature of between −10° C. and 70° C.

6. The process of claim 5, wherein the temperature is between 0° C. and 10° C.

7. The process of claim 1, wherein said inert, organic solvent is a chlorinated aliphatic hydrocarbon, an aromatic hydrocarbon, a dialkyl ether or a carbon disulfide.

8. The process of claim 7, wherein the inert, organic solvent is selected from the group consisting of: chloroform; dichloromethane; 1,1-dichloroethane; 1,2-dichloroethane; 1,1,2-trichloroethane; 1,1,2,2-tetrachloroethane; 1,2-dichloroethylene, 1,1,2-trichloro-ethylene; benzene; toluene; nitrobenzene; chlorobenzene; diethylether; and di-n-propylether.

9. The process of claim 8, wherein the solvent is either chloroform or dichloromethane.

10. The process of claim 1, wherein said catalyst is selected from the group consisting of:
a) a halogen, an oxide or an oxo acid of an element of Group Va, VIa, or VIIa of the periodic table, or a salt thereof; and
b) an oxide of an element of Group VIII of the periodic table, or a salt thereof.

11. The process of claim 10, wherein said catalyst is selected from the group consisting of:
a) elemental chlorine, bromine or iodine;
b) an alkali, earth alkali or ammonium salt of the corresponding hydrohalic acid of elemental chlorine, bromine or iodine;
c) a mixture of any of the elements of a) and/or the salts of b); and
d) osmium tetroxide.

12. The process of claim 1, wherein said catalyst is elemental iodine.

13. The process of claim 1, wherein the molar ratio of oxidizing agent to beta-carotene or a carotinoid is 1:1 to 10:1 mol.

14. The process of claim 13, wherein said molar ratio is 1:1 to 5:1 mol.

15. The process of claim 1, wherein oxidation is carried out at a pH of 4 to 8.

16. The process of claim 1, wherein the reaction time is 0.1 to 10 hours.

17. The process according to claim 1, wherein the reaction time is 1 hour.
